(19) United States
(12) Patent Application Publication
(10) Pub. No.: US 2005/0084512 A1
(43) Pub. Date: Apr. 21, 2005

(54) Tridimensional biocompatible support structure for bioartificial organs and uses thereof

(76) Inventors: Francine Denizeau, Verdun (CA); Mircea Alexandru Mateescu, Montreal (CA); Yascara Grisel Luna Saavedra, Montreal (CA)

Correspondence Address: ROBIC, 55 ST-JACQUES, MONTREAL, QC, H2Y 3X2, CA

(21) Appl. No.: 10/688,941

(22) Filed: Oct. 21, 2003

(51) Int. Cl. A61F 2/00

(52) U.S. Cl. 424/423; 424/93.7

(57) ABSTRACT

The present invention relates to a biocompatible support structure for culturing cells in three dimensions. In a preferred embodiment, the support structure is constituted essentially of cross-linked polyvinylalcohol (PVA). More preferably, the matrix has the form of a sponge and is used for the culture of hepatocytes. The invention also relates to methods of manufacturing such structure and to methods of using the same in vitro, ex vivo as well as in vivo. The invention further relates to a bioartificial organ and to a tridimensional cell culture system which may be used for the production of therapeutic proteins, used as a detoxification device, used as a tool in predictive toxicology of compounds in the pharmaceutical industry and/or used for transplantation.

### BACKGROUND OF THE INVENTION

A) Field of the Invention

The present invention relates to a biocompatible support structure for culturing cells in three dimensions. The invention also relates to methods of manufacturing such structure and to methods of using the same in vitro, ex vivo as well as in vivo, particularly as a bioartificial organ.

B) Brief Description of the Prior Art

Over the last decade, there has been a continuously growing interest in new polymeric matrices for growing cells and tissues. However, despite the multitude of polymeric matrices that have been developed (e.g. hydrogels, hydrophilic or hydrophobic matrices), the mechanical characteristics and compatibility with the cultured tissues must still be improved. In this context, special attention was paid to hydrogels and sponges based on polymeric materials, which are already accepted in various therapeutic applications. They consist of hydrophilic macromolecules cross-linked to form a swellable but insoluble three-dimensional network. As examples, such hydrophilic polymers include hydroxypropyl-methylcellulose [HPMC], ethylcellulose, acyl-substituted cellulose and vinylic polymers such as poly(hydroxyethylmethacrylate)-PHEMA, polyacrylamides, other acrylic copolymers, polyvinylacetate (PVAc), polyvinylpyrollidone (PVP), polyesters, and many other derivatives that are used as pharmaceutical excipients. Polymer swelling and the permeability of solutes (i.e., metabolites) through hydrogels depend on the chemical structure of the polymer (composition, molecular weight, cross-linking density and crystallinity as well as on the type of the agent to be transferred), which can modulate the viability of the system.

Various substrata have been used for the culture of hepatocytes in vitro based upon natural as well as synthetic materials. Natural materials such as laminin, fibronectin, entactin, collagen, perlecan, glycoproteins, proteoglycans and MATRIGEL® have the advantage of being capable of specific interactions with the cells, but their availability is extremely limited and their costs very high, making them inapplicable when large quantities are needed. Other natural polymers such as alginates do not have the same limitations. However, they lack motifs for specific interactions with the cells, and their properties cannot be easily modified to compensate for this severe drawback.

Synthetic materials have been shown to be useful for controlled drug release tablets. For instance, U.S. Pat. No. 5,456,921 describes the use of cross-linked amylose as a matrix for the slow release of biologically active compounds. Similarly, interpenetrated CL-HAS and CL-PVA networks CL (HAS-PVA) have been produced and used as excipients for controlled drug release tablets (U.S. Pat. No. 6,284,273).

Synthetic materials have also been shown to be useful for in vitro cell culture, including: polyhydroxymethylmetacrylate, poly-L-lactic acid, polyglycolic acid, poly-N-p-vinylbenzyl-d-lactonamide, polycaprolactone, cross-linked polyurethane, polyvinylformal and polyvinylalcohol. These polymers offer interesting advantages because they can be synthesized in large quantities in a reproducible manner and at low cost. Moreover, their mechanical and chemical properties can be modified and improved, so that they are enriched in specific information providing optimal cell signaling and performance.

Despite the efforts that have been devoted so far to the development of a bioartificial organ such as bioartificial liver, the existing models all suffer from important limitations. They all show a lack of performance regarding at least one of the key features in optimal system design, which include: the tridimensional structure of the substratum, the availability and cost of the tridimensional substratum, the potential for enriching the tridimensional substratum in specific information for cell anchorage and programmed signaling, and the ease of perfusion of the cell-substratum phase.

There is thus a need a tridimensional culture system that circumvents the above-mentioned limitations.

There is also a need for a biocompatible support structure for culturing cells and growing tissues in three dimensions and which offers improved mechanical properties, better biocompatibility with cultured cells and tissues, and a higher stability against enzymatic or microbial degradation.

There is further a long felt need for bioartificial organs such as a bioartificial liver.

There is a need also for improved ex vivo methods for producing therapeutic proteins, for the detoxification of body fluids, and for evaluating the toxicity and biological activity of compounds in the pharmaceutical industry.

The present invention fulfils these needs and also other needs which will be apparent to those skilled in the art upon reading the following specification.

### SUMMARY OF THE INVENTION

An object of the invention is to provide a support structure that can be used as a matrix for cell and tissue growing, and which offers improved mechanical properties, better biocompatibility with cultured cells and tissues, and a higher stability against enzymatic or microbial degradation than existing cell culture systems.

Therefore, according to two related aspects, the invention relates to a biocompatible support structure for culturing cells in three dimensions and to bioartificial organs that mimic the in vivo microenvironment of chosen cells.

Even though, in the following description, different examples of biocompatible support structures and bioartificial organs are disclosed, the present invention as claimed hereinafter is restricted to a biocompatible support structure for culturing cells in three dimensions, which support structure comprises a biocompatible and non-biodegradable polymeric material on which cells may adhere and proliferate, and which forms, when saturated in a suitable aqueous medium, a porous tridimensional sponge-like scaffold with a plurality of interconnected pores, the pores being dimensioned and distributed so that a flow of at least 0.1 ml/min⁻¹ cm⁻² of an aqueous solution may circulate through said biocompatible support structure. This biocompatible support structure as claimed is characterized in that the polymeric material consists of a cross-linked polyvinylacohol (PVA) derivatized with alkylamino groups.

According to another aspect, the invention relates to a practical, efficient and economic tridimensional cell culture system for the culture of mammalian cells, and more particularly human hepatocytes, that solves several problems that could not be solved by the conventional monolayer culture such as loss of liver-specific functions, dedifferentiation and cell mortality.

The bioartificial organs and culture system of the invention may be used for the production of therapeutic proteins, used as a detoxification device, used as a tool in predictive toxicology of compounds in the pharmaceutical industry and/or used for transplantation.

An advantage of the present invention is that it provides a tridimensional support structure for cell culture that is easy to manipulate, that is biocompatible, non-biodegradable and which shows stable mechanical and chemical properties in vitro as well as in vivo. The support structure of the invention further comprises interconnected channels that allow cell-cell communication, a very important feature for cell survival. It is also possible to modify the polymeric matrix and/or to incorporate thereto bioactive molecules in order to improve cell-matrix interactions, cell-cell interactions and matrix-recipient interactions.

According to one aspect, the invention relates to a tridimensional cell culture system in which the cells remain viable and are capable of maintaining a number of specific functions over a long period of time, for instance albumin secretion for liver cells.

Other objects and advantages of the present invention will be apparent upon reading the following non-restrictive description of several preferred embodiments, made with reference to the accompanying drawings.

### BRIEF DESCRIPTION OF THE DRAWINGS

FIGS. 1A and 1B are confocal microscopy images of hepatocytes adhering to the support structure of the invention after 72 hours of culture. FIG. 1A: General view of a preferred embodiment (highly porous PVA matrix) showing interconnected pores (bar=500 μm). FIG. 1B: Hepatocytes (green) are shown making cell-cell and cell-matrix interactions (bar=100 μm). Propidium iodide was used as a red marker for the PVA matrix and 5-chloromethylfluoroescein diacetate as a green marker for live hepatocytes.

FIG. 2 is a diagram showing a preferred embodiment of a tridimensional culture system according to the present invention.

FIG. 3 is a graph showing the percentage of hepatocytes adherent to different types of support structures under static culture conditions. Abbreviations: M: disc of PVA-matrix unmodified; MM: disc on PVA-matrix modified by aminoethylation both being tridimensional static cultures; and F: collagen film in monolayer culture.

FIG. 4 is a graph showing the percentage of lactate dehydrogenase released over time by hepatocytes cultured on different supports and under static culture conditions. The abbreviations are the same as in FIG. 3.

FIG. 5 is a graph showing the adherence of hepatocytes on different supports and under dynamic culture conditions. Abbreviations: cM: cylinder-shaped PVA polymer-matrix without any modification; cMM: cylinder-shaped PVA polymer-matrix modified with aminoethylation, both being tridimensional supports under dynamic culture conditions; and F: collagen film in monolayer culture as control.

FIG. 6 is a graph showing the percentage of lactate dehydrogenase released by hepatocytes cultured on different supports and under dynamic culture conditions. The abbreviations are the same as in FIG. 5.

### DETAILED DESCRIPTION OF THE INVENTION

The present invention relates to a biocompatible support structure for culturing cells in three dimensions, consisting essentially of a biocompatible and non-biodegradable polymeric material on which cells may adhere and proliferate.

As used herein, “biocompatible” means compatible and relatively favorable to cell attachment and proliferation, and without overly negative effects onto these two cell-related functions. As used herein, “non-biodegradable” means substantially resistant to chemical degradation or enzymatic digestion which occurs normally in biological systems. For the purposes of the present invention, resistance to chemical degradation or enzymatic digestion is long enough so that the support structure performs its desired function(s) (typically a few days), but it may be longer (weeks, months, years).

The invention also relates to methods of manufacturing such a biocompatible structure and to methods of using the same in vitro, ex vivo as well as in vivo, particularly as a bioartificial organ.

Preferred cells to be cultured on the support structure of the invention are mammalian cells and more preferably human cells. Preferred cells include but are not limited to hepatocytes, cardiomyocytes, fibroblasts, osteoblasts, cancer cells, monoclonal cells, kidney cells, and pancreatic cells such as Langerhans cells and acinar cells. However, other types of cells could also be used depending on a user's intended uses. Indeed, the origin of the cells can be diverse depending on the application sought (cell line, cells purified from a patient, cells that have been genetically modified, etc).

A) Tridimensional Support Structure

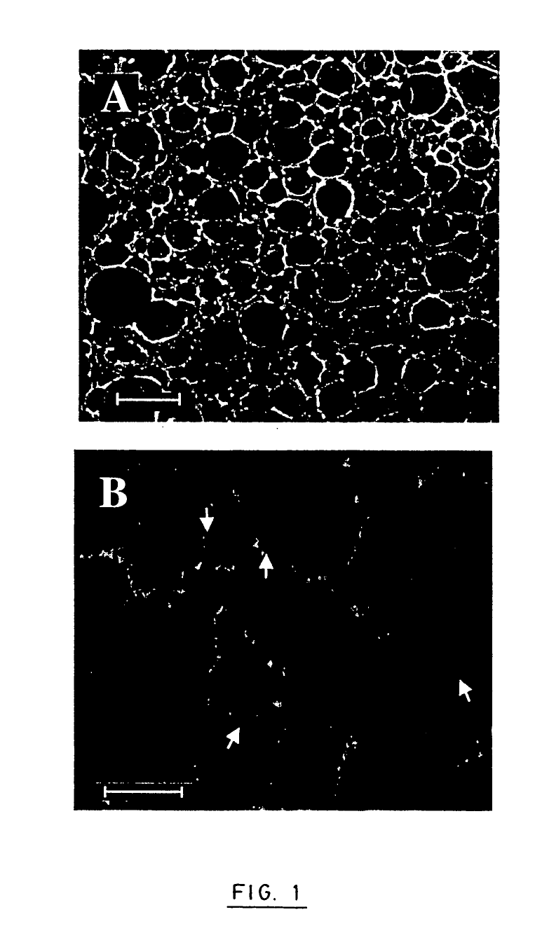

According to a first aspect, the invention relates to a biocompatible support structure (also called herein a matrix) for culturing cells in three dimensions. In a preferred embodiment, the support structure consists essentially of a biocompatible and non-biodegradable polymeric material on which cells may adhere and proliferate (see FIGS. 1A and 1B). The support structure forms, when saturated in a suitable aqueous medium, a porous tridimensional sponge-like scaffold with a plurality of interconnected pores that are dimensioned and distributed so that a flow of at least 0.1 ml/min⁻¹ cm⁻², preferably 0.5 ml/min⁻¹ cm⁻², and more preferably of 1 to about 15 ml/min⁻¹ cm⁻², of an aqueous solution may circulate through the biocompatible support structure. In preferred embodiments, the support structure comprises from about 20 to about 37 pores/cm² and the pores have a diameter of about 100 to about 1000 μm.

More preferably, the biocompatible and non-biodegradable polymeric material of which is made the support structure is selected from the group consisting of polyvinylalcohol (PVA), polyuretans, polycyanoacrylates, polyhydroxyethyl methacrylate (PHEMA), methylmethacrylate (MMA), and N-vinyl pyrrolidone (N-VP). Even more preferably, these compounds are treated with cross-linking agents like epichlorohydrin, sodium trimetaphosphate, POCL₃, formaldehyde (HCHO) and 2,3 dibromophenol.

In a most preferred embodiment, the biocompatible support structure consists essentially of a polyhydroxylic polymer with alternate polar and non-polar groups. More preferably, the support structure consists essentially of cross-linked polyvinylalcohol (PVA) molecules, including but not limited to amino-ethyl-polyvinylalcohol (AE-PVA), arginyl-glycyl-aspartyl-polyvinylalcohol (RGD-PVA) and derivatives thereof.

PVA is a material that possesses interesting characteristics that can be exploited for tridimensional cell culture. The present inventors have found that a PVA-derived support structure according to the present invention exhibits better mechanical properties and improved biocompatibility, when compared with matrices made of other polymers used in prior art. The modified polymeric PVA chains preserve their tridimensional structure over a long period, since PVA is articulated by: a) multipoint interchain covalent bridges introduced by a cross-linking reaction, and b) by interchain hydrogen bonding contributing to network stabilization.

PVA is a polyhydroxylic polymer containing alternate polar (—CH—OH) and non-polar (—CH₂—) groups. This represents a well-balanced structure in terms of chain polarity, and at the same time, enough flexibility for interchain interactions and particular macromolecular conformations. Based on its crystallinity, a helical structure was assumed for PVA. It appears that a syndiotactic configuration favors both interchain hydrogen association and helical structure. Consequently, this conformation can lead to a higher water-resistance of PVA.

PVA also exhibits fewer polar (—OH) groups per repetitive unit —[CH₂—CH(OH)—]ₙ—, than carbohydrates. With a hydrophobic —CH₂— residue, which gives a relative flexibility of the rotation towards the whole polymeric chain, in alternating with the hydroxylic one [—CH(OH—)], which is hydrophilic and can be involved in hydrogen associations, PVA can generate particular helix structures. Cross-linked PVA (CLPVA) can also generate a crystalline network. The tendency to reach an advanced order (interchain stabilization by hydrogen bonding) is a common structural characteristic for several carbohydrates (amylose, agarose) and PVA polymers which are susceptible to form helices. This characteristic is important for network formation. Their reticulation and derivatization can be perfectly compatible with stable structures and adequate properties.

The present inventors also observed that, biocompatible and non-biodegradable polymeric materials comprising an amine function demonstrated an increased adherence of hepatocytes on their surfaces. Therefore, according to a preferred embodiment of the invention, cell attachment capabilities of the support structure are increased by adding amine functions (or related peptides ensuring better cell adhesion) on the surface of the biocompatible and non-biodegradable polymeric material. This can be achieved by modifying the biocompatible and non-biodegradable polymeric material (preferably PVA) with haloalkyl amines. A non-restrictive list of suitable haloalkyl amines includes 2-chloroethylamine hydrochloride, chloropropyl amine, bromoethylamine, iodoethylamine. More preferably, 2-chloroethylamine hydrochloride is used.

The biocompatible support structure of the invention may further comprises an associated polymer incorporated to the structure in order to enhance its possibilities. The associated polymer(s) may constitute from about 1 to about 50% w/w of the structure and it is selected according to its compatibility with the biocompatible and non-biodegradable polymeric material and according to its usefulness in cell culture. For instance, if the support structure is to be used ex vivo in a bioartificial organ such as a prosthesis, it would be preferable that the polymeric material(s) incorporated to the structure be biocompatible, but not easily biodegradable. For in vivo transplantation uses, it would be preferable that the associated polymer(s) incorporated into the structure be biodegradable. A non-restrictive list of suitable associated polymers includes polyethyleneglycol (PEG), agarose, starch, alginate, and chitosan.

As it will be explained with more details hereinafter, the biocompatible support structure may also further comprises a bioactive molecule in order to increase the cells attachment and/or cells proliferating capabilities of the structure.

In view of the above, one can see that the highly porous and biocompatible support structure of the invention presents numerous beneficial characteristics. Indeed, when saturated in aqueous medium, the support structure preferably takes the form of a porous tridimensional structure similar to that of marine sponges with interconnected pores that are of comparable size and of variable forms. The support structure has a swelling volume that is very flexible and it has a high mechanical resistance. It is thus capable of resisting to temperatures up to 90° C. without showing any plastic deformation. The support structure of the invention may also resists against the action of diluted acids, strong alkali, solutions of common detergents, and most organic solvents.

B) Manufacture of the Tridimensional Support Structure

The support structure of the invention is preferably synthesized according to the specific needs of a user. Although PVA is a highly preferred biocompatible and non-biodegradable polymeric material according to the invention, the present invention is not limited only to PVA-derived matrices. Indeed, PVA is a prototype polymer with regards to the applications that are described herein. The concept of the invention applies to other polymers sharing with PVA one of many desired properties such as biocompatibility, the capacity to form a porous structure with interconnected pores of controlled dimension; the presence of anchoring groups on the polymer, such as hydroxyl groups, that can serve to add specific biological functions. The biocompatible and non-biodegradable polymeric material is selected according to a desired use and desired characteristic properties of the final product. A non-exhaustive list of polymers that fulfills at least some of the previous requirements has been given hereinbefore.

Interestingly, it was found that a PVA-polymeric sponge bought from PVA Unlimited Company (Warsaw, Ind., USA) possessed most of the above mentioned desired characteristics. Such sponge may be cut out according to a desired shape and use (preferably in the form of a cylinder having a diameter of about 1 to 10 cm and a length of about 1 to 50 cm).

Although such a commercially available sponge is preferred, similar sponges having substantially the same desired characteristics could also be used and/or manufactured. For instance, a tridimensional PVA-derived support structure may be manufactured by cross-linking PVA molecules with bifunctional agents such as epichlorohydrin, as described in U.S. Pat. No. 5,456,921 or with other agents such as 2,3 dibromopropanol, sodium trimetaphosphate, phosphorous oxichloride, linear anhydrides of di-carboxylic acids, diepoxides, dialdehydes, phosgene, imidazolium salts of polycarboxylic acids, cyanuril chloride and derivatives thereof.

In a more preferred embodiment, the support structure consists essentially of cross-linked polyvinylalcohol (PVA) molecules, including but not limited to amino-ethyl-polyvinylalcohol (AE-PVA), arginyl-glycyl-aspartyl-polyvinylalcohol (RGD-PVA) and derivatives thereof. Such cross-linked polyvinylalcohol (PVA) molecules may be obtained by polymer treatment with a cross-linking agent such as epichlorohydrin, 2,3-dibromopropanol, sodium trimetaphosphate, formaldehyde, glutaraldehyde and other functional aldehydes. Preferably, the cross-linking degree of the support structure is adjusted between about 1% to 10% since it is known that high cross-linking generates a reduction of chain flexibility which is required for hydrogen association. However, for other types of materials, the cross-linking degree may be of up to 50%. Once a tridimensional support structure having desired selected features has been synthesized, it is preferably washed and sterilized before use.

As mentioned previously, the support structure of the invention may also be enriched with various types of bioactive molecules in order to optimize and/or increase cell-matrix interactions, cell-cell interactions and matrix-recipient interactions and/or to improve the cells attachment and/or cells proliferating capabilities of the structure.

A non-restrictive list of examples of interesting bioactive molecules includes:
signaling molecules and extracellular biocompatible matrix proteins (e.g., fibronectin, laminine) or motifs of these proteins such as peptides where the RGD sequence (Arg-Gly-Asp) is present;
peptide binding motifs of receptors involved in cell-cell interactions (e.g. integrines, cadherines);
carbohydrates or carbohydrate derivatives such as glycosaminoglycans (heparin, heparan sulfate), N-glycans, sialic and polysialic acid, sialyl sugars, carbohydrate dendrimers, sulfated glycoconjugates; and
growth factors or hormones or active components of such signaling molecules (e.g. EGF, HGF).

Methods for linking bioactive molecules to PVA or equivalent polymers are numerous. Examples are as follows:

a) Methods to Link Peptides Directly to a Polyhydroxylic Polymer:
polymer activation with cyanogen bromide (BrCN) and peptide binding via amino groups;
polymer activation with p-benzoquinone and peptide binding via amino groups;
polymer reaction with trichloro-S-triazine (cyanuric chloride) and peptide binding via amino groups;

b) Methods to Link Peptides on Polymeric Matrices Derivatized with Carboxyl Functions:

Support structure exhibiting carboxyl groups can be obtained by derivatization with carboxyalkyl groups, following treatment with ε-chloroalkyl carboxylic acids X—(CH₂)ₙ—COOH where: X═Cl, Br, I and n=1-12 and more. As an example (not limitative), carboxymethyl (CM)-PVA structures obtained by treatment of PVA with monochloroacetic acid can be considered. The carboxylic functions can be involved in:
reactions with carbodiimides and amino groups of peptides;
reaction with SOCl₂ and amino groups of peptides;
reaction with Woodward reagent (activation of carboxyl functions with N-ethyl-5-phenylisoxazolium3′-sulfonate) and peptide binding via amino groups;
reaction with azide (treatment of CM-matrices with hydrazine and then with NaNO₂) and peptide binding via amino groups.

c) Methods to Link Peptides to Polymeric Matrices Derivatized with Amino Functions:

Support structure can be obtained by derivatization with aminoalkyl groups, following treatment with X—(CH₂)ₙ—NH₂ where: X═Cl, Br, I and n=1-12 and more. As an example (not limitative), can be considered the aminoethyl (AE)-PVA matrices obtained by PVA treatment with chloroethylamine. The amino group can be involved in:
reaction with carbodiimide and peptide binding via carboxyl groups;
reaction with dialdehydes (i.e. glutaraldehyde) and peptide binding via amino groups (with a Schiff base as intermediate);
reaction with COCl₂ (carbonic dichloride) and peptide binding via amino groups.

d) Methods to Link Peptides to Polymeric Matrices Derivatized with Thiol (SH) Functions:

Polymeric matrices can be obtained by derivatization with thioalkyl groups, following treatment with X—(CH₂)ₙ—SH where: X═Cl, Br, I and n=1-12 and more. As an example (not limitative), can be considered the thiopropyl (SH)-PVA matrices obtained by PVA treatment with chloropropylthiol. The thiol function can be involved in reaction of HS-PVA with 2,2′-dipyridyl disulfide; the PVA-S-pyridyl disulfide obtained will bind Cys-peptides (i.e., CRGD) via their HS-(Cys) groups.

Methods to obtain a controlled release of active agents (i.e. specific peptides modulating cell adhesion, growth factors, etc.) from the support structure (or matrix) of the invention can also be envisaged. It could be possible for instance to load a PVA-derivatized matrix according to the invention with selected active agents by swelling the PVA matrix in the presence of selected active agent(s), then drying the PVA-gel obtained. When cell culture is initiated, the active agent would preferably be released by diffusion through the matrix or by controlled lysis of specific links. It may be also desirable to allow a bio-erosion of the matrix over the cell-culturing period by conjugating together the active agent and the polymers of the matrix with hydrolyzable bonds. Such bonds may be: esters, amidic functions, particular carbohydrate linkages, and the like.

C) Uses of the Tridimensional Support Structure of the Invention

The biocompatible support structure of the present invention has many applications for instance in sutures, in drug delivery systems (e.g. microencapsulated islet of Langerhans for the treatment of diabetes), in tissue engineering (e.g. implants, cartilage replacement, artificial skin, nerve reconstruction), in artificial cells (e.g. red-blood-cell substitutes), as a bioartificial organ (e.g. liver, kidney, pancreas), in gene therapy (encapsulation of genetically modified cells), and in ex vivo methods for producing therapeutic proteins, for the detoxification of body fluids, and for evaluating the toxicity and biological activity of compounds in the pharmaceutical industry.

C-1) Bioartificial Organ

According to another aspect, the invention relates to a bioartificial organ, comprising: i) a biocompatible support structure as defined previously, and ii) living cells which are adhered and which are proliferating on the support structure. As used herein, the term “bioartificial organ” include also “bioreactor”.

Bioartificial organs and/or bioreactors incorporating the matrix of the invention could be particularly useful for: transplantation purposes (e.g. hepatocyte transplantation, liver regeneration), the production of prosthesis, the production of therapeutic proteins, ex vivo body fluids detoxification, or testing new pharmaceutical compounds.

Preferably, the bioartificial organ consists of a bioartificial liver, a bioartificial kidney or of a bioartificial pancreas. According to a most preferred embodiment, the bioartificial organ consists of a bioartificial liver comprising from about 1 million to about 50 million hepatocytes/cm³ (more preferably about 25 to 30 million/cm³) attached and proliferating onto a support structure made of PVA.

As it will be explained with more details hereinafter, the bioartificial organ of the invention may be used for carrying out the following methods:
a method for culturing cells in three dimensions in vitro;
a method for producing therapeutic proteins in vitro;
a method for detoxifying biological fluids ex vivo;
a method for assaying in vitro the toxicity, biological activity or pharmaceutical activity of drugs; and
a method for prolonging the life of a patient in need of an organ transplantation.

More particularly, a bioartificial liver according to the invention has numerous applications such as:
production of therapeutic proteins normally synthesized by the liver (e.g. albumin, ceruloplasmin, and clotting factors);
use as an ex vivo detoxification device for patients suffering acute intoxication with drugs or toxins;
use as an in vitro tool in predictive toxicology for early pre-clinical evaluation and optimization of lead compounds in the pharmaceutical industry (predictive toxicology or drug metabolism); and
use as bioartificial liver in acute liver failure for bridging with transplantation.

Therefore, according to another related aspect, the invention relates to a method for prolonging the life of a mammal. The method comprises the steps of providing a bioartificial organ as defined previously, and introducing this bioartificial organ in the body of a mammal in need thereof in replacement of a malfunctioning organ. Preferably, bioartificial organ is a bioartificial liver, a bioartificial kidney or a bioartificial pancreas.

According to another related aspect, the invention relates to a method for manufacturing a bioartificial organ comprising a plurality of cells. The method comprises the steps of:
isolating desired cells from a mammal;
introducing these cells into a suitable aqueous culture system comprising (i) a suitable culture medium for the in vitro or ex vivo culture of said cells, and (ii) a biocompatible support structure as previously; and
generating a suitable flow of the culture medium and of the cells through the biocompatible support structure for a sufficient period of time and under conditions for allowing cells to adhere to the biocompatible support structure and replicate therein. The circulation of culture media is preferably continuous, but is may also be pulsed.
   C-2) Method, Device and System for Cells Culture

According to a further aspect, the invention relates to a method for culturing cells. The method comprises the steps of:
providing a suitable aqueous culture system comprising: (i) a suitable culture medium, (ii) a biocompatible support structure as defined previously, and (iii) cells for which culture is desired;
generating a suitable flow of the culture medium and of the cells through the biocompatible support structure for a sufficient period of time and under conditions for allowing cells to adhere to the biocompatible support structure and replicate therein; and, optionally
recovering in the flow of culture medium, molecules produced by the cells.

Preferably, the cells consist of hepatocytes, cardiomyocytes, fibroblasts, osteoblasts, cancer cells, monoclonal cells, kidney cells, and pancreatic cells. Therefore, recovered molecules produced by the cells may be human therapeutic proteins (e.g. albumin, ceruloplasmin, insulin, clotting factors, growth factors, monoclonal antibodies, hepatic enzymes, pancreatic enzymes and intestinal enzymes. Of course, the cells may be genetically modified in order to produce such human therapeutic proteins or in order to increase a basal level of secretion.

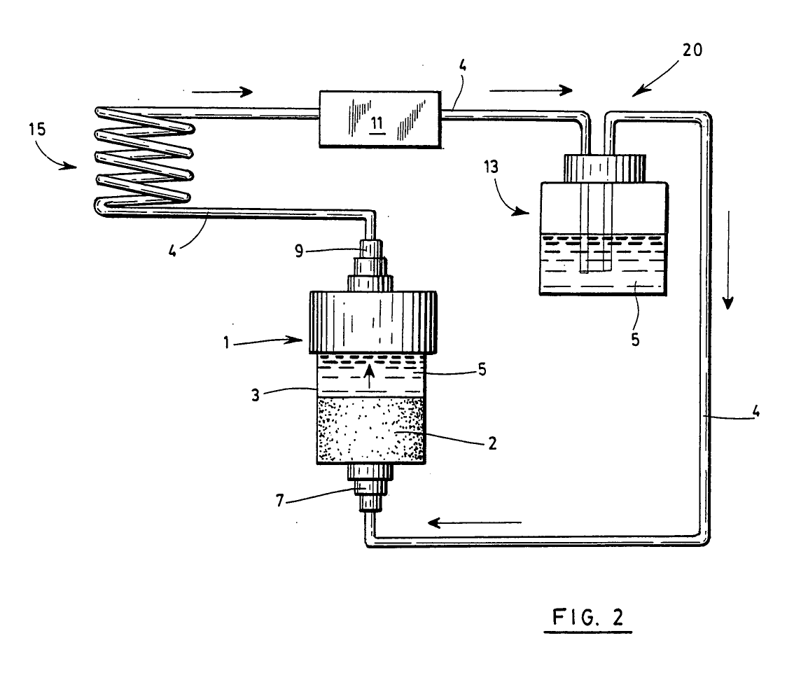

According to a related aspect, the invention relates to a device for culturing cells in three dimensions. A preferred embodiment of a culture system comprising such a device is shown at FIG. 2. More particularly, the device (1) comprises a cylindrical waterproof housing (3) through which a culture medium (5) can circulate. The housing (3) has an inlet (7) and an outlet (9) capable of a waterproof connection to pumping means (11). The device (1) also comprises a biocompatible support structure (2) as defined previously that is enclosed into the waterproof housing (3).

According to another related aspect, the invention relates to a tridimensional system for cell culture and tissue growing. A preferred embodiment of a culture system according to the invention is shown at FIG. 2. The tridimensional cell culture system (20) of the invention comprises:
cells (not shown) for which culture in three-dimension is desired;
a culture medium (5) that is suitable for the in vitro or ex vivo culture of the cells;
a cell culture device (1), as defined previously, for culturing the cells; and
pumping means (11) for circulating the culture medium though the cell culture device (1).

Preferably, the pumping means (11) have control means to adjust a flow of culture medium circulating through the device and through the biocompatible support structure. The circulation of culture media is preferably continuous, but it may also be pulsed.

More preferably, the tridimensional cell culture system (20) further comprises an oxygenator (15) for injecting oxygen in the system (preferably 95% O₂ and 5% CO₂). Preferably also, the system (20) further comprises a reservoir (15) filled with culture media (5) for insuring a minimum level of media in the system. The cell culture device (1), the oxygenator (15), the pumping means (11), the reservoir (15) are connected with biocompatible tubes (4).

According to a preferred embodiment, the support structure is put in a suitable culture medium and under suitable culture conditions with the cells to be cultured. Preferably, the cells and the support structure are put in a dynamic cell culture system which allows a continuous flow of medium through the matrix and cells are seeded in sufficient quantities so that proper cell-cell contacts are established. The culture system to be used must allow physiological exchanges between the cells and the extracellular fluid. It is also highly preferable that the composition and the circulation of the medium through the cellular compartments be carefully controlled. Well-known culture systems and bioreactors which have been used in various studies on cell physiology, metabolism and morphology, in pharmacology and toxicology, as well as in the development of bioartificial organs include: BAL™ device, Hollow Fiber Bioreactor, CELLMAX FLOW MODULE™, rotating-wall vessels (RWV), Flat Membrane bioreactor (FMB). In all these systems, optimal culture conditions (pH, % CO₂, % O₂, T°) may be maintained for long periods of time.

According to a preferred embodiment of the invention, the dynamic cell culture system comprises a CELLMAX FLOW MODULE™ (CELLCO™, Spectrum Laboratories Inc., Laguna Hills, USA) that has been adapted, by the present inventors, to be used with the matrix of the invention. More particularly, a cylindrical glass cartridge of 5.1 cm in length and 2.1 cm in diameter was designed to fit the PVA sponges. Once in culture the cells attach gradually to the matrix, and after a certain period of time, they typically colonize most of the internal and external surfaces of the matrix. Depending on the desired uses, the culture period should last such that the matrix entraps an adequate number of cells, preferably from about 1 million to 25 million of cells/cm³.

C-3) Ex Vivo Method for Detoxification of Blood

According to another related aspect, the invention relates to a method for detoxifying ex vivo blood of a mammal from undesirable chemicals or toxins such as acetominophene, metronizadol, antitumoral agents (e.g. methotrexate, mercaptopurine), poisons (e.g. acetone, chloroform), drugs (e.g. cocaine, heroine) etc. The method comprises the steps of:
providing a bioartificial organ, preferably bioartificial liver, as defined previously, the bioartificial comprising cells with detoxifying capabilities;
circulating through the bioartificial organ blood of the mammal under ex vivo conditions such that the cells of the bioartificial organ detoxicate circulating blood from undesirable drugs or toxins.
   C-4) Method for in Vitro Assessment of Toxicity, Biological Activity, and/or Pharmacological Activity of a Compound

According to another related aspect, the invention relates to a method for assaying in vitro the toxicity, biological activity, and/or pharmacological activity of various compounds such as known or potential pharmaceutical compounds, carcinogenic compounds, toxic compounds (e.g. toxins, solvants), carcinogenic compounds, drugs (e.g. cocaine, heroine) etc. The method comprises the steps of:
providing a compound, of a plurality of compounds, for which assessment of toxicity or biological activity is desired;
providing a bioartificial organ as defined previously, the organ comprising cells for which compound(s) assessment is desired;
contacting the compound(s) with the bioartificial organ;
optionally circulating through the bioartificial organ a suitable culture medium for the in vitro or ex vivo culture of the cells; and
evaluating the toxicity, biological activity, and/or pharmacological activity of the compound(s) on the cells of the bioartificial organ.

A non-restrictive list of suitable cells useful for carrying out the method of the invention includes hepatocytes, cardiomyocytes, fibroblasts, osteoblasts, cancer cells, monoclonal cells, kidney cells, and pancreatic cells.

The toxicity, biological activity, and/or pharmacological activity of the compound(s) may be done using well-known methods, techniques or tools. For instance, one may easily measure intracellular or secreted levels of proteins (e.g. albumin or lactate dehydrogenase released by hepatocytes), cell survival, mortality and/or functions.

### EXAMPLES

As a proof of concept, hepatocytes were inoculated on modified or unmodified polymer PVA matrices. The results obtained and shown hereinafter clearly indicate that PVA-derived matrices have a great potential for the tridimensional culture of cells. Indeed, it was observed that polymers treated with 2-chloroethylamine hydrochloride and having ethylamine functions in their chemical composition demonstrate an increased adherence of hepatocytes on their surfaces.

It is to be understood however that the following examples are illustrative of the wide range of applicability of the present invention and are not intended to limit its scope. Modifications and variations can be made therein without departing from the spirit and scope of the invention. Although any methods and materials similar or equivalent to those described herein can be used in practice for testing of the present invention, the preferred methods and materials are described.

A) Introduction

The liver performs numerous complex functions. It is involved in the metabolism of carbohydrates, lipids, amino acids and proteins. It is responsible for glycogenolysis and gluconeogenesis, the synthesis of lipoproteins and cholesterol, and the synthesis of essential proteins such as albumin, transferrin and clotting factors. The liver plays a prominent role in the biotransformation of xenobiotics and in detoxification processes. It is the site of bile production and the storage of essential nutrients including retinol, folic acid and cobalamin. Failure of the liver to carry out its normal functions as a result of liver disease leads to a life-threatening condition.

Death by liver disease occurs mainly in two clinical settings: cirrhosis and fulminant hepatic failure (FHF). Cirrhosis is the end stage of liver disease characterized by the replacement of normal tissue with fibrotic tissue, culminating in the confinement of the remaining hepatocytes, which no longer have the capacity to regenerate. Cirrhosis develops in alcoholism and chronic hepatitis. FHF is an extremely severe disease with a mortality rate of more than 80%. Its major cause is hepatitis following chemical intoxication or viral infection. Because the onset of FHF is sudden and its progression very rapid, death can occur within only a few weeks after the symptoms have appeared. Liver dysfunction is accompanied by increased serum liver enzymes, ammonia and bilirubin, as well as by decreased serum albumin and clotting factors. Ultimately, encephalopathy sets in, but the exact mechanisms responsible for this complication are still unknown.

Liver disease is a worldwide major problem. In North America, over 20 millions people are stricken by liver disease, which leads to the death of more than 40 000 individuals each year. Liver cirrhosis is the seventh leading cause of death by disease, affecting several hundred million people around the world. In the United States, hepatitis C virus (HCV)-related liver disease is the single most common cause for liver transplantation. In Canada, increased incidence of liver disease due to viral infection is anticipated, as it is estimated that at least 1% of the population is infected with HCV, a figure that clearly shows endemic proportions. The country that is hardest hit by HCV is Egypt, where it has been evaluated that roughly 24% of the population carries HCV.

The management of liver failure represents a formidable therapeutic challenge. Liver transplantation appears to be the long-term solution. In spite of this, there is a severe lack of well-preserved livers available for transplantation. In 1996 in the United States, 4 058 liver transplant operations were performed, but it has been estimated that up to 10 000 potential recipients died because of organ scarcity. The deficit in liver availability has become more pronounced in the last 10 years. In the United States, from 1988 to 1996, the number of liver donors increased 2.4-fold, while the number of patients on the waiting list for a transplant soared from 616 to 7467 (over 10 fold), and the number of deaths resulting from lack of proper treatment quintupled.

In view of the unavailability of organs at the critical stage of liver failure, there is an urgent need to develop systems which could provide “bridging” support to the patient awaiting a donor organ, thus significantly increasing chances of survival. Procedures bypassing the necessity of transplantation altogether might also even be possible. At the present time, efforts are being devoted to designing artificial liver tissue/organ systems in order to find a viable solution to end-stage liver disease therapy. The objective of providing bioartificial liver therapy requires that these systems be engineered so that the hepatocytes function properly, and therefore, provide the capacity to save the life of a patient undergoing liver failure.

The microenvironment of the cells in a bioartificial organ is of prime importance for expression of their specific functions. For hepatocytes, there is a close relationship between the tridimensional architecture of the cells and their differentiation. Morphological integrity and intercellular communication are associated with the proper biochemical regulation of hepatocytes. Optimal conditions for the culture of hepatocytes require that the tri-dimensional organization of the cells in vitro mimic the organization seen in vivo as closely as possible. In an in vitro culture system for hepatocytes, the topography of the tridimensional substrate can have its own effects on the cells, influencing cellular polarization, cytoskeletal arrangement and functional activity. In addition to the information generated by the physical microenvironment, biochemical components of the milieu are of key importance in producing signals that promote cell differentiation and function.

We are now proposing new polymeric materials based on the cross-linking of polyvinylalcohol (PVA), followed by its derivatization with specific alkyl-amino and/or peptide groups. These materials with higher compatibility can be particularly useful as matrices for cells and tissues growing for bioartificial livers and other organs. Theses polymeric materials were of interest, having stable mechanical and chemical properties that could be useful for further surface modifications. Considering that the number of cell-cell and cell-matrix interactions is very important in maintaining differentiation, liver-specific functions and hepatocyte viability, the characteristics of that polymer were tested in order to determine whether a polymeric PVA sponge and other similar materials could be optimized for providing a suitable microenvironment for the tridimensional culture of hepatocytes.

The PVA matrix of the invention shows better mechanical properties since this polymer can be stabilized by: i) covalent cross-linking and ii) via hydrogen bonding. PVA chains are known to be easily stabilized by hydrogen bonds (with calculated length of 4.5-5.7 Å and confirmed by X-ray measurement). Only a proper modification of PVA can ensure proper mechanical properties. For instance, only a moderately low density of covalent epichlorohydrin cross-linking bridges (calculated at 8.4 Å) allows enough flexibility of the chains to induce their self-assembly by hydrogen bonds (4.5 Å). This is why PVA exhibits interesting particular properties, related to the degree of cross-linking.

B) Material and Methods

Preparation of Collagen Coated Dishes

Type I collagen was obtained from rat tail tendon by the procedure of Michalopoulos and Pitot (Exp Cell Res. 94: 70-78, 1975). Plates were prepared by distributing 1.5 ml of the collagen solution (approximately 0.75 mg/60 mm tissue culture dishes); they were then kept overnight in a sterile atmosphere.

Isolation and Culture of Rat Hepatocytes

Hepatocytes were isolated from male Sprague Dawley rats (150-300 g) by a modification of the collagenase perfusion technique of Seglen (Meth Cell Biol. 13: 29-83, 1976) as described in Guillemette et al. (Biochem Cell Biol 71: 7-13, 1993). Briefly, the liver was first perfused via the portal vein with 400 ml of a calcium-free Hanks' buffer solution supplemented with 0.25 mM of EGTA for 15 minutes and then perfused with a second Hanks' buffer solution containing 100-200 U/ml of collagenase (Sigma-Aldrich) with 5 mM of calcium hydrochloride for 8 minutes to digest the liver tissue. The perfusates were equilibrated with 95% O₂ and 5% CO₂. Then the perfused liver was resected and settled in a glass dish containing cold Williams Medium E (WME, Sigma-Aldrich)+10% bovine fetal serum (FBS) (Gibco Life Technologies) fresh medium. Then the capsule was slowly teased apart and the lobes were delicately shaken to recover the hepatocytes. The resulting suspension was filtered through two nylon meshes with grid sizes of 250 μm and 50 μm. The cell pellet was collected by centrifugation at 50 g for 2 minutes. Viable hepatocytes were isolated from cellular debris and non-parenchymal liver cells using a Percoll™ (Amersham Pharmacia Biotech) gradient centrifugation at 4° C. at 1000 g for 8 minutes. The cell pellet was washed once with WME+10% FBS. Hepatocyte viability was determined by flow cytometry (FACSCAN™, Becton Dickinson) with propidium iodide (2 mg/ml). To examine how hepatocytes function on the PVA polymer support, we performed measurements of liver-specific functions.

Culture Conditions

PVA-matrices in static conditions were placed in 60 mm culture Petri dishes to which 5 ml of culture medium was added. A modified cartridge housing the polymer was secured vertically to the CELLMAX FLOW MODULE™ (CELLCO™, Spectrum Laboratories Inc., Laguna Hills, USA) for the constructs in flow conditions. It was exposed to a 5 ml/min (lowest speed of the module) continuous flow of L-15 (Leibovit'z L-15 ) culture medium (Meth Cell Biol. 13: 29-83, 1976) pumped from a 100 ml reservoir (total volume of medium was 60 ml) and recirculated back to the reservoir after passing through the module (See FIG. 2). The flow module and the cartridge were placed in an incubator and cultures were performed at 37° C. with 5% CO₂. The media was changed every day for both the static and dynamic cultures.

Polymer Seeding

Constructs (PVA-discs fitting to 60 mm petri dishes) under static culture conditions were seeded with 2×10⁶ hepatocytes/disc. Constructs (PVA-cylinder shape 1.5 cm in diameter and 2.0 cm high) under dynamic culture conditions were seeded with 3.0×10⁷ hepatocytes/cylinder by injection inoculation.

Aminoethylation of PVA-Matrices

Polyvinylalcohol (PVA) sponges were purchased from PVA Unlimited Company (Warsaw, Ind., USA). The surface modifications were done as follows. Sponges were dried for 48 hours in an oven at 40° C. Then the dry weight of sponges to be modified was determined. The sponges were then hydrated and covered with a minimum of water at 70° C. Everything was placed in a 70° C. water-bath. An amount of 2-chloroethylamine hydrochloride (Fluka Chemika-Biochemika) equal to the weight of the polymer material was dissolved in a minimum of water and added to the sponges minimal volumes of 10 M NaOH were added to keep the reactional medium at pH=9.0 for a 2-hour reaction time. Finally, the modified sponges were washed five times in distilled water and dried for 48 hours at 40° C.

Determination of the Albumin Secretion of Hepatocytes Cultured under Different Conditions

Every day, the culture medium was collected from the different systems in Eppendorf tubes and kept at −20° C. until assayed. The rate of albumin secretion from the hepatocytes was quantified by a sandwich enzyme-linked immunosorbent assay (ELISA) using rat-specific antibodies. Briefly, 96-well plates (FALCON™, Becton Dickinson) were coated (200 μl/well) with 1 μg/mL of anti-rat albumin antibody (Nordic Cederlane Laboratories) diluted with 0.1 M Na₂CO₃ buffer solution, pH=9.6 and left overnight at 4° C. After washing the plates three times with a phosphate buffer saline (PBS)/0.05% FBS (Gibco Life Technologies) (200 μl/well) for 30 minutes at 37° C., the plates were blocked with a PBS/0.05% FBS (Gibco Life Technologies) solution for 30 minutes at 37° C. After three washes with the PBS buffer solution, pH=7.3, 200 μl of the diluted (dilution solution: PBS/0.05% TWEEN™/0.5% bovine serum albumin (BSA, previously shown not to give any cross-reaction with rat anti-albumin antibodies used for ELISA), Sigma-Aldrich, ICN Biomedicals) samples and standards (chromatograhically purified albumin, Sigma-Aldrich) was deposited in triplicate on the plates and incubated for 1 hour at 37° C. After three washings with a PBS/0.05% TWEEN™ solution pH=7.3, peroxidase-conjugated anti-rat albumin antibody (1 μg/ ml) diluted in PBS/5% FBS solution was added and incubated for 1 hour at 37° C. After three washings with PBS-0.05% TWEEN™ solution, 200 μl of substrate (0.5 mg/ml o-phenylenediamine, Sigma-Aldrich) diluted in 0.1M phosphate citrate buffer pH=5 with 0.006% H₂O₂ was added to each well, and the optical density at 490 nm was measured with a microplate TITERTEK MULTISKAN™ reader.

Viability Determination of Hepatocytes

Lactate dehydrogenase (LDH) activity was determined in the media as a measure of hepatocyte deterioration. For this study, the culture medium was harvested daily and the 24-hour release of LDH into the medium was quantitated. Total LDH levels in the cells were also determined for each culture. For the LDH assay, 28 μl of the sample was added to each well on a 96-well plate. To obtain the value representing 100% LDH release, cells were treated with 1% Triton 10× for 10 minutes. Then 250 μl of the reagent for LDH assay (0.12 M NaCl, 4.83 mM KCl, 0.96 mM KH₂PO₄, 1.96 mM MgSO₄.7H₂O, 2.5 mM CaCl₂.2H₂O, 23.8 mM NaHCO₃, 2% BSA, 6.8 mM Na pyruvate, 1 mM NADH, pH=7.4) was added to each well and the optical density was immediately read at 340 nm with a microplate TITERTEK MULTISKAN™ reader. All measurements were done in triplicate.

Determination of Cellular Adherence to Different Matrices

The cellular adhesion on the supports was determined by counting the number of cells recovered in the culture medium. For the initial count, after the adherence period, the cells were counted as follows: Number of inoculated cells−Number of cells found in the medium=Number of cells that remained attached to the substratum.

Thereafter, cells were counted daily as follows: Number of cells that remained attached to the substratum at the previous day−Number of cells found in the medium=Number of cells that remained attached to the substratum at the day of measurement.

C) Results

### EXAMPLE 1

### Preliminary Trials on Bidimensional Cultures

Table 1 hereinafter shows best adhesion properties for aminoethyl derivatives of different polymers studied.

TABLE 1
Composition, mechanical and cell adhesion properties
of different polymeric films and derivatives used as
possible matrices for hepatocytes culture.

| | Mechanical | Adhesion |
|---|---|---|
| Films and chemical compositions | stability | property |
| CM-CLA (6)-CLPVA-6 | + | + |
| CMCL6-CLPVA6 + 5% collagen | + | + |
| CL (A-PVA)6 | − | Nd |
| CL (A-PVA)6 + 5% collagen | − | Nd |
| AECLA/PropylCLA/CMCLA6 1/1/1 + 2% epi | + | ++ |
| CMCLA6 + CLPVA3/1 + 2% epi + 5% | + | Nd |
| collagen | | |
| AECLA/PropylCLA/CMCLA6 1/1/1 | + | ++ |
| Agar | +++ | − |
| ⅓ AECLA-0/Agar | +++ | + |
| ½ AECLA-0/Agar | +++ | Nd |
| 3/2 AECLA-0/Agar | +++ | Nd |
| AECLA-0 | +/− | +++ |
| AECLA-6 | +/− | +++ |
| 1% agarose | +++ | − |
| 1% agarose + ⅓ AECLA-0 | +++ | ++ |
| 1% agarose + ½ AECLA-0 | +++ | ++ |
| 1% agarose + 2/1 AECLA-0 | +++ | Nd |
| Chitosan + 5% collagen | + | ++ |

Legend:
+++ = excellent,
++ = good,
+ = low,
+/− = unstable,
− = none,
Nd = not defined.
Abbreviations:
CM, CarboxyMethyl;
PVA, polyvinylalcohol;
AE, aminoethyl;
epi, epichlorohydrine (cross-linking agent);
CLA, cross-linked amylose n (0-6) = cross-linking degree, defined as the amount of cross-linking agent (g) used to cross-link 100 g of polymer.

### EXAMPLE 2

### Tridimensional Hepatocytes Culture on PVA-Polymer Support under Static Conditions

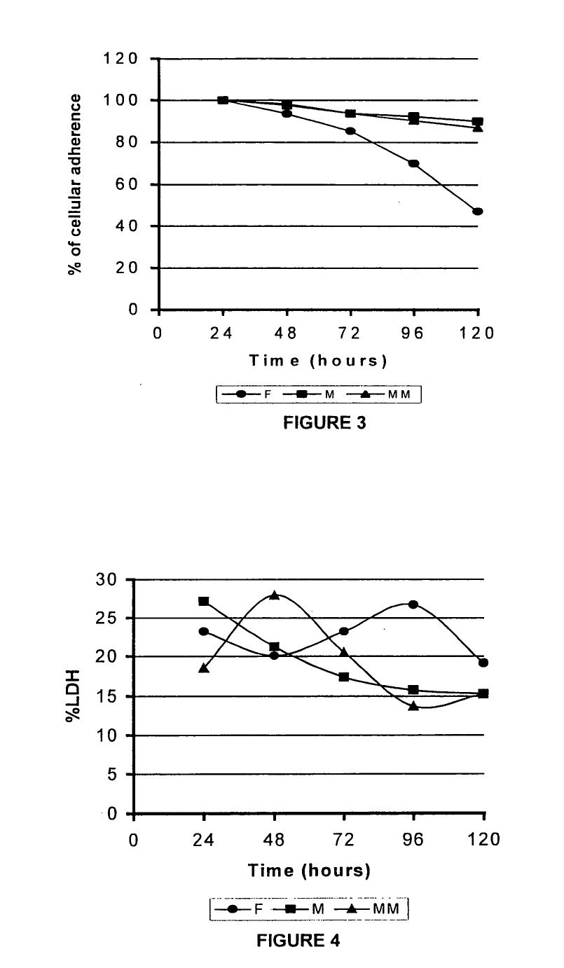

FIG. 3 shows better maintenance of cell adhesion on PVA and AE-PVA matrices as compared to collagen film.

Table 2 shows higher albumin secretion with AE-PVA as compared to collagen film.

FIG. 4 shows stabilization of cell viability with PVA matrices after 72 h in culture.

TABLE 2
Metabolic activity of hepatocytes under static culture conditions.

| | Albumin secretion (μg/10⁶ cells/24 h) | | | | | |
|---|---|---|---|---|---|---|
| | Time (hrs) | | | | | |
| | 24 | 48 | 72 | 96 | 120 | Activity retention % |
| F | 26.3 | 19.2 | 12.8 | 4.8 | 2.3 | 8.6 |
| M | 28.8 | 20.3 | 8.0 | 12.3 | 4.1 | 14.2 |
| MM | 27.5 | 25.1 | 12.4 | 9.4 | 4.9 | 17.8 |

Abbreviations:
M, disc of PVA-matrix unmodified;
MM, disc on PVA-matrix modified by aminoethylation both being tridimensional static cultures; and
F, collagen film in monolayer culture.

### EXAMPLE 3

### Tridimensional Hepatocytes Culture on PVA-Polymer Support under Dynamic Conditions

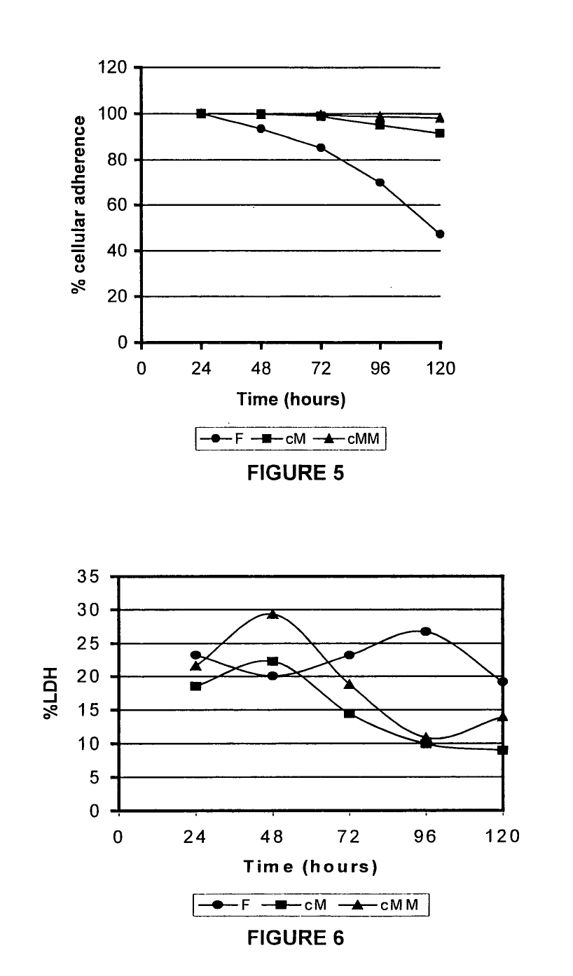

FIG. 5 shows better maintenance of cell adhesion on PVA and AE-PVA matrices under constant perfusion.

Table 4 shows higher albumin production with PVA and AE-PVA matrices under perfusion conditions as compared to collagen film (cf. Table 2).

FIG. 6 shows stabilization of cell viability with PVA matrices after 72 h in culture under perfusion conditions.

TABLE 4
Metabolic activity of hepatocytes in dynamic culture conditions.

| | Albumin secretion (μg/10⁶ cells/24 h) | | | | | |
|---|---|---|---|---|---|---|
| | Time (hrs) | | | | | |
| | 24 | 48 | 72 | 96 | 120 | Activity retention % |
| cM | 28.7 | 16.2 | 7.8 | 9.1 | 7.2 | 25.2 |
| cMM | 27.8 | 18.8 | 12.8 | 12.3 | 6.1 | 22.0 |

Abbreviations:
cM, cylinder-shaped PVA polymer-matrix without any modification; and
cMM, cylinder-shaped PVA polymer-matrix modified with aminoethylation, both being tridimensional supports under dynamic culture conditions.

D) Discussion

Hepatocytes were seeded under two different culture conditions: 1) in a static system, on collagen film (F) or on a PVA synthetic polymer disc (M), or on a PVA-aminoethyl modified polymer disc (MM); 2) in a dynamic system: on a PVA synthetic polymer cylinder-shaped matrix (cM) or on PVA-aminoethyl modified polymer cylinder-shaped matrix (cMM).

The quantification of albumin secretion (Tables 2 and 4) showed that hepatocytes cultured under dynamic conditions retained a superior percentage of secretion activity after 120 hours, relative to day 1, than hepatocytes cultured under static conditions. Moreover, hepatocytes cultured on tridimensional supports under dynamic or static conditions showed a better final retention of albumin secretion function (relative to initial activity at day 1) compared to hepatocytes seeded on bidimensional monolayer culture support.

FIGS. 4 and 6 show the percentage of LDH release in the culture medium as a measure of loss of hepatocyte viability in culture. We observed that cultures on collagen film show higher quantities of free LDH than cultures where hepatocytes were seeded on PVA-polymer matrices.

The results in FIGS. 3 and 5 indicate that the number of adherent cells on the collagen film decreases rapidly with time. In contrast, the number of hepatocytes seeded on the PVA-polymer matrices is almost stable over time, showing the efficiency of cell adhesion on a tridimensional microenvironment. This support is more adapted to maintaining the seeded hepatocytes because there are more interactions between the matrix and the cultured cells.

In summary, these results confirm that tridimensional polymers offer a better extracellular matrix environment for the adhesion of hepatocytes. Also, the results confirm that a perfusion system increases the beneficial properties of tridimensional culture: it maintains optimal conditions for the survival of hepatocytes and preserves albumin secretion activity.

While several embodiments of the invention have been described, it will be understood that the present invention is capable of further modifications, and this application is intended to cover any variations, uses, or adaptations of the invention, following in general the principles of the invention and including such departures from the present disclosure as to come within knowledge or customary practice in the art to which the invention pertains, and as may be applied to the essential features hereinbefore set forth and falling within the scope of the invention or the limits of the appended claims.

1. A biocompatible support structure for culturing cells in three dimensions, which comprises a biocompatible and non-biodegradable polymeric material on which cells may adhere and proliferate, and which forms, when saturated in a suitable aqueous medium, a porous tridimensional sponge-like scaffold with a plurality of interconnected pores, said pores being dimensioned and distributed so that a flow of at least 0.1 ml/min⁻¹ cm⁻² of an aqueous solution may circulate through said biocompatible support structure, characterized in that said polymeric material consists of a cross-inked polyvinylalcohol (OVA) derivatized with alkylamino groups.

2. The biocompatible support structure of claim 1, wherein said pores are dimensioned and distributed so that a flow of at least 0.5 ml/min⁻¹ cm⁻² of an aqueous solution may circulate through the biocompatible support structure.

3. The biocompatible support structure of claim 2, wherein said pores are dimensioned and distributed so that a flow of about 1 to about 15 ml/min⁻¹ cm⁻² of an aqueous solution may circulate through the biocompatible support structure.

4. The biocompatible support structure of claim 1, wherein said pores have a diameter of about 100 to about 1000 μm.

5. The biocompatible support structure of claim 1, wherein it comprises from about 20 to about 50 pores/cm².

6. The biocompatible support structure of claim 1, wherein said cross-linked polyvinylalcohol (PVA) is derivatized by reacting its hydroxyl functions with an haloalkyl amine.

7. The biocompatible support structure of claim 6, wherein said haloalkyl amine is selected from the group consisting of 2-chloroethylamine hydrochloride, chloropropyl amine, bromoethylamine and iodoethylamine.

8. The biocompatible support structure of claim 1, wherein said support structure further comprises an associated polymer selected from the group consisting of polyethyleneglycol (PEG), agarose, starch, alginate, and chitosan.

9. The biocompatible support structure of claim 1, wherein said support structure further comprises a bioactive molecule selected from the group consisting of: extracellular biocompatible support structure proteins, growth factors, hormones, signaling molecules, peptide binding motifs of receptors, carbohydrates, and carbohydrates derivatives.

10. The biocompatible support structure of claim 1, wherein said cells consist of mammalian cells.

11. The biocompatible support structure of claim 10, wherein said mammalian cells consist of human cells.

12. The biocompatible support structure of claim 1, wherein said cells are selected from the group consisting of hepatocytes, cardiomyocytes, fibroblasts, osteoblasts, cancer cells, monoclonal cells, kidney cells, and pancreatic cells.

13. A bioartificial organ, comprising:
a biocompatible support structure; and
living cells which are adhered and which can proliferate on said support structure,
wherein said support structure is as defined in claim 1.

14. The bioartificial organ of claim 13, wherein said living cells consist of mammalian cells.

15. The bioartificial organ of claim 14, wherein said mammalian cells consist of human cells.

16. The bioartificial organ of claim 13, wherein said living cells are selected from the group consisting of hepatocytes, cardiomyocytes, fibroblasts, osteoblasts, cancer cells, monoclonal cells, kidney cells, and pancreatic cells.

17. The bioartificial organ of claim 16, wherein said bioartificial organ consists of a bioartificial liver, a bioartificial kidney or a bioartificial pancreas.

18. A device for culturing cells of the type comprising:
a waterproof housing through which a culture medium can circulate, the housing having an inlet and an outlet capable of a waterproof connection to pumping means; and
a biocompatible support structure that is enclosed into said waterproof housing;
characterized in that said biocompatible support structure is as defined in claim 1.

19. A tridimensional cell culture system comprising:
cells for which culture in three-dimension is desired;
a culture medium that is suitable for the in vitro or ex vivo culture of said cells;
a device for culturing cells as defined in claim 18; and
pumping means for circulating a culture medium though said device.

20. The system of claim 19, characterized in that said cells are selected from the group consisting of hepatocytes, cardiomyocytes, fibroblasts, osteoblasts, cancer cells, monoclonal cells, kidney cells, and pancreatic cells.
